(19) United States
(12) Patent Application Publication
(10) Pub. No.: US 2016/0106747 A1
(43) Pub. Date: Apr. 21, 2016

(54) TREATING POSTOPERATIVE MECHANICAL STRESS WITH AN ECTOINE

(71) Applicant: Bitop AG, Witten (DE)

(72) Inventors: Rene Tolba, Aachen (DE); Gerhild van Echten-Deckert, Bonn (DE); Georg Lentzen, Dortmund (DE); Andreas Bilstein, Witten (DE)

(73) Assignee: Bitop AG, Witten (DE)

(21) Appl. No.: 14/980,374

(22) Filed: Dec. 28, 2015

Related U.S. Application Data
(63) Continuation-in-part of application No. 13/707,259, filed on Dec. 6, 2012.
Application No. 13/707,259 is a continuation of application No. 12/863,832, filed on Sep. 22, 2010, now abandoned.

(30) Foreign Application Priority Data
Jan. 30, 2008 (DE) 102008006780.6

(51) Int. Cl. A61K 31/505; A61K 9/00; A61K 9/08

(52) U.S. Cl. CPC A61K 31/505; A61K 9/08; A61K 9/0048

(57) ABSTRACT

A method of treatment of a patient suffering from postoperative inflammatory stress and pain caused by mechanical impact exerted on portions of the body of the patient that causes damaged tissue wherein the inflammatory stress and pain of the operation outlast the healing of damaged tissue and which is not related to, or caused by, uncontrolled proteolysis. The treatment comprises administering a tetrahydropyrimidine selected from ectoine and/or hydroxyectoine to the patient.

### CROSS-REFERENCE TO RELATED APPLICATIONS

This application is a continuation-in-part of, and claims the benefit of, U.S. patent application Ser. No. 13/707,259, filed Dec. 6, 2012, which is a continuation of, and claims the benefit of, U.S. patent application Ser. No. 12/863,832 filed Sep. 22, 2010, which claims the benefit of PCT/EP2009/000641, filed Jan. 30, 2009.

### FIELD OF THE INVENTION

The invention relates to tetrahydropyrimidines and their use in the prevention and treatment of postoperative inflammatory stresses and pain.

### BACKGROUND OF THE INVENTION

Low-molecular organic chemical compounds isolated from extremophilic organisms have a remarkable influence on biological systems and structures. Such compounds are termed osmolytes or compatible solutes and have meanwhile found their way into numerous cosmetic preparations.

Aside from sugars, sulfur compounds, polyols and amino acids especially tetrahydropyrimidine derivatives such as ectoine and hydroxyectoine count among compatible solutes. They are synthesized from extremophilic microorganisms under stress conditions and serve the purpose of stabilizing the cell structures of these microorganisms under extreme thermal, chemical and physical conditions. Examples in this respect are halophilic microorganisms that must adapt to the changing salt content in a saline environment and must survive in this environment.

Ectoine and its derivatives are known to have a stabilizing effect on protein and nucleic acid structures with said effect being conducive to the stabilization of biological material and pharmaceutical preparations.

As regards ectoine and its derivatives a number of medical preparations have been described in the meantime, for example with respect to their use as dermatologic agent as well as to raise the efficiency of protein containing active agents. Moreover, a therapeutic effect has provably been found in the treatment of endothelial function disturbances.

US. Publication 2003/0014356 to Galinaki et al. (“Galinski”) describes the use of compatible salutes including ectoine and hydroxyectoine among others to stabilize biopolymers against degradation by enzymes, in particular by proteases. U.S. Pat. No. 6,180,607 to Davies describes proteolysis as leading to a myrad of diseases, using polynucleotides to inhibit proteolysis. Bunnet (Seminars in Thrombosis and Hemostasis, 21 (Sup 1):39-48, 2006), cited in prosecution of the parent of this application, describes the protease that activates protease activated receptors as being generated and secreted during injury and inflammation.

The protecting effect of proteases is linked to a change in structure of the biopolymer caused by the compatible solute so that the degrading enzyme no longer recognizes the target. Galinski also suggests making use of this effect with tissue injuries, wound healing, acute and chronic inflammation, and graft rejection, among others. It is the specific protease inhibiting activity of compatible salutes that is made use of by Galinsk. Proteolysis is associated with the healing process of a wound and ends with disappearance of damaged tissue. It does not play a role where there is long lasting stress and pain. Long lasting means that the negative effects outlast the healing process for a long time, in sonic cases permanently.

Proteolysis does not play a role in eye surgery, where muscular tissue is not affected. It is known that both eye surgery, in particular LASIK treatment, and abdominal surgery, e.g. in connection with cancer treatment, frequently result in long lasting stress and pain. Abdominal surgery is frequently associated with pain attacks, eye surgery with dryness that causes a sandpaper-like feeling.

### BRIEF SUMMARY OF THE INVENTION

In view of the known physiological effects of ectoines the now detected effectiveness in connection with postoperative inflammatory stress and pain frequently encountered as the result of surgical interventions, in particular in the abdominal cavity, has come as a major surprise. The invention relates to a method of treatment of postoperative inflammatory stress and pain in a class of patients suffering from postoperative inflammatory stress that doesn't suffer from uncontrolled proteolysis, comprising administering a tetrahydropyrimidine selected from ectoine and/or hydroxyectoine to a patient in need of such treatment. More particularly, a method of treatment is provided for a patient suffering from postoperative inflammatory stress and pain caused by mechanical impact exerted on portions of the body of the patient that causes damaged tissue wherein the inflammatory stress and pain of the operation outlast the healing of damaged tissue. The stress and pain related to, or caused by, uncontrolled proteolysis, ends with disappearance of damaged tissue, it does not outlast the healing of damaged tissue. The treatment comprises administering a tetrahydropyrimidine selected from ectoine and/or hydroxyectoine to the patient.

The cause and nature of the stress and pain conditions is not entirely understood. One assumption is that the healing process results in a re-arrangement of tissue and membranes which also effects the innervation and pain sensation. Tissue re-arrangement results in shear and tension on a molecular level which cause friction and irritation. Ectoines are known to enhance water accretion to biological tissues and membranes. This tissue hydration is known to stabilize the helical structure of proteins and provides a gliding effect that reduces such irritation. After eye surgery, ectoines provide a beneficial liquid film with their hydration ability.

### BRIEF DESCRIPTION OF THE DRAWINGS

For a more complete understanding of the present invention, reference is now made to the following descriptions taken in conjunction with the accompanying drawing, in which:

FIG. 1 shows the values in U/L determined for the lactate dehydrogenase (LDH)-liberation in samples treated with POLYSOL only (P) and those to which ectoine was added (E);

FIG. 2 depicts the liberation of NOₓ⁻ in mmol/L for the samples treated with POLYSOL (P) and those to which ectoine (E) was added; and

FIG. 3 shows electron microscope images of tissue samples after a 30-minute reperfusion with oxygen, in POLYSOL (P) and with ectoine (E). AM denotes the apical cell membrane, MV the microvilli which are in much better condition in the samples treated with ectoine.

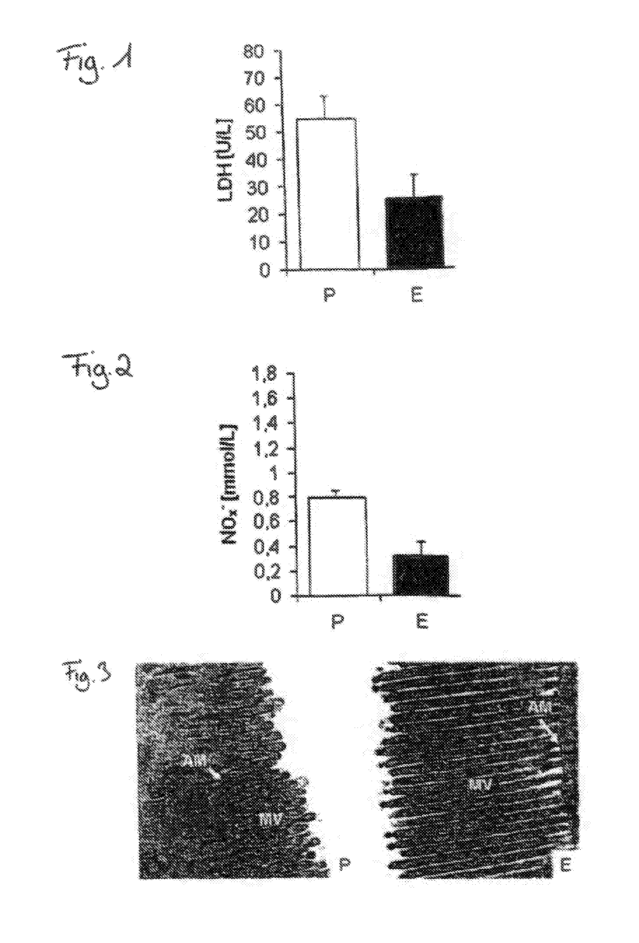

### DETAILED DESCRIPTION OF THE INVENTION

Accordingly, the invention relates to tetrahydropyrimidines of the formula

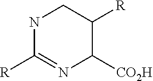

where R is a hydrogen atom or a hydrocarbon radical comprising up to eight carbon atoms, R′ is hydrogen, OH or OR″ and R″ is R or COR, with R having the above described significance for use in the prevention or treatment of postoperative inflammatory stresses and pain, in particular in the abdominal and intestinal region.

Operations or interventions within the meaning of the invention are any interventions that cause the body to suffer mechanical stresses, for example also in conjunction with endoscopic examinations, also in the area of joints or in the abdominal region. In particular, said interventions are surgical interventions of a significance greater than those that merely cause a punctiform opening/penetration of the skin.

In conjunction with surgical interventions so-called postoperative inflammatory stress and pain are frequently experienced that may give rise to major problems the patient has to cope with. Frequently enough, inflammatory stress and pain are not the result of the surgical intervention as such but are rather due to inflammation phenomena caused by the mechanical impact exerted on portions of the body which are not the target of the operation, for example when the abdominal cavity, especially the gastrointestinal region, but also liver and kidneys are involved in surgery, and if endoscopic examinations are conducted. Such a mechanical exposure is encountered in the abdominal cavity, for example, where it may be necessary to displace intestinal loops during the operation, make enlargements to the abdomen or, especially when examinations are carried out, apply pressure to the intestine itself or abdomen. The inflammation phenomena resulting from these activities and the inflammatory stresses and pain accompanying them may occasionally continue for a long time after surgery. This is also the case when operations are performed in other regions of the body, for instance during dental extractions, jaw surgery or interventions necessary to treat fractures.

The same applies to transplantations, in particular in the region of the abdomen. If, for example, intestinal segments are transplanted inflammatory reactions as described above often delay the healing process, cause major rejection problems and even complete failures. Exploratory investigations have shown that ectoines and related compounds are conducive to counteracting these inflammatory phenomena and pains caused by surgery.

For the purposes of the invention ectoines are (4S)-1,4,5,6-tetrahydropyrimidine-4-carboxylic acids and their derivatives. The tetrahydropyrimidine carboxylic acids may have a hydrocarbon group comprising up to eight carbon atoms at 2-position, for example an alkyl group or a methyl group. Moreover, at 5-position the tetrahydropyrimidine may be substituted by a hydroxy group, in particular by a (5S) hydroxy group. The hydroxy group may be etherified or esterified so as to be pharmaceutically acceptable.

Preferred ectoines are ectoines themselves, (4S)-2-methyl-1,4,5,6-tetrahydropyrimidine-4-carboxylic acid and hydroxyectoine, (4S, 5S)-5-hydroxy-2-methyl-1,4,5,6-tetrahydropyrimidine-4-carboxylic acid.

The invention proposes that several ectoines may be applied together. As derivatives those shall be considered that essentially have the same or better effects than the relevant basic substance.

The tetrahydropyrimidines proposed by the invention may, of course, be combined with the customary adjuvants and auxiliary substances. The agents used in this context may be provided for oral or parenteral administration, for example in the form tablets, capsules or as solution. The agents may be administered in one or several doses with the doses ranging between 1 and 250 mg per kg of body weight and day, preferably 5 to 150 mg, and in particular 10 to 100 mg.

Aside from an oral or parenteral administration the agent may also be administered topically, for example in the form of an irrigation of the intestinal region or abdomen to be treated with a pharmaceutically acceptable solution, for example a physiological solution containing the tetrahydropyrimidine. Due to the excellent physiological compatibility of tetrahydropyrimidines which are fully soluble in water such irrigation solutions may contain up to 25% of the tetrahydropyrimidine. However, this content will as a rule amount to 0.5 to 5%.

According to the invention tetrahydropyrimidines are especially suited as well for the administration and application in conjunction with the POLYSOL solution furnished by Doorzand Medical Innovations, said solution being used for irrigation and preservation, also of transplants. Tetrahydropyrimidine in the range of between 0.01 and 10% w/w may be added to the POLYSOL solution, in particular approx. 0.1 to 1%.

The use of tetrahydropyrimidines as proposed by the invention shall not be deemed to be limited to postoperative inflammatory phenomena, stress and pain. On the contrary, their field of use in fact embraces surgical interventions in virtually any part of the body and they are favorably employed for practically any type of surgery. This covers, among others, dental extractions, jaw surgery and implantations, also of teeth, artificial joints, eye operations and more.

Another area of application is to use tetrahydropyrimidines in connection with ischemic reperfusion occurring in conjunction with the transport and/or treatment and/or implantation of organs. It has been found that through the use of tetrahydropyrimidines and in particular ectoines not only the negative effects stemming from operations as explained above may be alleviated but at the same time implant healing chances may improve significantly and the healing time span be shortened.

For the purpose of closer investigation an animal model involving rats was prepared to induce enteritis by administering 10 mg/kg of trinitrobenzol sulfonic acid in 50% ethanol using a light ether anesthesia. Lesions developed which were examined macroscopically four days later. Ectoine was administered orally in various doses (30 to 300 mg/kg) for one week before the rats were given TNBS and four days thereafter. It could be seen that ectoine was capable of alleviating the extent of the lesions, and the maximum effect could be achieved with a dose of 100 mg/kg.

From a biochemical viewpoint, ectoine prevented changes of the myeloperoxidase activity and caused the glutathione level in the large intestine to go down. Ectoine, moreover, had a protective effect in that it prevented changes of the level of various mediators, including IKAM-1, DNFα, IL-1β, IL-10, LTB₄ and PGE₂ both in the blood and in the tissue of the large intestine. The effect was found to be comparable to that of sulfasalazine with 300 mg/kg, which was used as reference active agent. The protective effect of ectoine could be confirmed on the basis of the histopathological examination of the large intestine.

### EXAMPLE

Small intestine segments of rats were removed under standard conditions and immediately irrigated with a cold common salt solution and subsequently with a POLYSOL solution (supplied by Doorzand Medical Innovations, Amsterdam/NL). The small intestine segments removed were kept ischemically in 50 ml of nutrient solution at 4° C. for 18 hours; in half of these cases ectoine (5 mg/kg) was added to the POLYSOL solution.

Following this storage time at 4° C. reperfusion of the small intestine segments took place in vitro at 37° C. using a modified KHB medium (5% Dextran 78, 0.95% KHB, 0.37 g/L CaCl₂, 2 g/L glucose, 0.6 g/L dexamethasone, 70 mg/L atropine and 0.21% sodium bicarbonate). For oxygen supply purposes a mixture of 95% O₂ and 5% CO₂ was used. The partial pressure of the oxygen was constantly kept at more than 500 mm/hg.

When reperfusion had been completed all tissue samples were deep-frozen at −80° C. in liquid nitrogen and examined to ascertain their condition, the lactate dehydrogenase (LDH) liberation and nitrite liberation as a measure of NO.

Regarding the LDH liberation as an indicator for the tissue quality values of 47.4+/−12.21 U/I and 54.5+/−8.57 U/I were detected after 15 and 30 min. for the samples preserved in POLYSOL. With respect to ectoine-modified POLYSOL, values of 8.8+/−3.42 U/I after 15 min. and 25.4+/−8.2 U/I after 30 min. were found. It can therefore be said that the use of ectoine reduces tissue damage and should thus have a positive influence on the healing process after surgery and transplantations.

Generally speaking, the formation of NOₓ is an indicator for (oxidative) stress to which a tissue sample is subjected.

In the control group containing POLYSOL treated samples an NOₓ⁻ formation of 0.78+/−0.063 mmol/L was found whereas in the control group with ectoine-doped samples values of 0.325+/−1.05 mmol/L were measured. Cutting NOₓ⁻ in half also means halving the formation of NO and as such reducing the oxidative stresses significantly.

Microscopic examinations of the tissue samples treated with ectoine have shown a considerably improved condition of the microvilli in contrast to the samples merely treated with POLYSOL.

1. A method of treatment of a patient suffering from postoperative inflammatory stress and pain caused by mechanical impact exerted on portions of the body of the patient that causes damaged tissue wherein the inflammatory stress and pain of the operation outlast the healing of damaged tissue and which is not related to, or caused by, uncontrolled proteolysis, comprising administering a tetrahydropyrimidine selected from ectoine and/or hydroxyectoine to the patient.

2. The method of claim 1, wherein the postoperative inflammatory stress and pain is pain in the abdominal and intestinal region.

3. The method of claim 1, wherein the postoperative inflammatory stress and pain is pain caused by transplantation in the region of the abdomen.

4. The method of claim 1, wherein the postoperative inflammatory stress and pain is stress and pain after endoscopic or laparoscopic interventions caused by inflammation.

5. The method of claim 1, wherein the tetrahydropyrimidine is hydroxyectoine.

6. The method of claim 1 wherein the tetrahydropyrimidine is administered to a patient in an amount of 1 to 250 mg per kg of body weight and day.

7. The method of claim 1 wherein the tetrahydropyrimidine is in the form of a solution for topical application comprising the tetrahydropyrimidine in an amount ranging between 0.1 and 25% w/w.

8. The method of claim 6, wherein the tetrahydropyrimidine is administered to a patient in an amount of 5 to 150 mg per kg of body weight and day.

9. The method of claim 6, wherein the tetrahydropyrimidine is administered to a patient in an amount of 10 to 100 mg per kg of body weight and day.

10. The method of claim 7, wherein the solution comprises 9.5 to 5% w/w of the tetrahydropyrimidine.

11. The method of claim 1 wherein the mechanical impact is from an eye operation.
